(19) United States
(12) Patent Application Publication
(10) Pub. No.: US 2024/0058267 A1
(43) Pub. Date: Feb. 22, 2024

(54) SLURRY AND SOLUTION COMPOSITIONS

(71) Applicant: MIRAKI INNOVATION THINK TANK LLC, Cambridge, MA (US)

(72) Inventors: Christopher VELIS, Lexington, MA (US); Karen MILLER, South Dartmouth, MA (US); Tarik S. CHAUDHRY, Boston, MA (US); Emilia JAVORSKY, Watertown, MA (US); William Roger MAILWARNING-BURTON, Cambridge, MA (US); Bradley Leo GUERTIN, Roseville, MN (US); Avi Aaron KURLANTZICK, Dedham, MA (US)

(73) Assignee: MIRAKI INNOVATION THINK TANK LLC, Cambridge, MA (US)

(21) Appl. No.: 18/140,124

(22) Filed: Apr. 27, 2023

Related U.S. Application Data
(63) Continuation of application No. 17/282,089, filed on Apr. 1, 2021, now abandoned.
(60) Provisional application No. 62/741,279, filed on Oct. 4, 2018.

(51) Int. Cl. A61K 9/10; A61K 9/00; A61K 33/14; A61K 47/38; A61M 5/32

(52) U.S. Cl. CPC A61K 9/10; A61K 9/0019; A61K 33/14; A61K 47/38; A61M 5/32

(57) ABSTRACT

Slurries comprise liquid water, about 2% to about 70% ice by volume, and one or more additives affecting flowability and/or tonicity of the slurry. Solutions for making a slurry comprise liquid water and one or more additives affecting flowability of the slurry. Flowability of the slurry relates to ice particles capable of flowing through a cannula, such as a needle. The slurry is suitable for injection into subcutaneous fat of a human subject for removal of adipose tissue.

### TECHNICAL FIELD

The invention relates to injectable slurry and solution compositions.

### BACKGROUND

Subcutaneous fat is present in varying amounts that generally correlate with genetic and lifestyle factors. Excess subcutaneous fat may impact health, fitness, and appearance. In many cases, individuals desire to reduce subcutaneous fat and have difficulty doing so through diet and exercise alone.

Conventional methods for subcutaneous fat removal, such as for surgical procedures like liposuction, are often painful, have long treatment duration, require a visit to a healthcare facility, and may have an extensive recovery period. In particular, traditional approaches may result in painful inflammation and discoloration at the treatment site.

Cryolipolysis refers to cold-induced reduction of adipose (fat) tissue. Given that lipid rich cells (such as subcutaneous fat and visceral fat) are more sensitive to cold injury than water-rich cells (such as skin and muscle), treatment of tissue with cool temperatures selectively targets fat cells and leaves other cell types unaffected. This concept of cryolipolysis has been used widely in devices that are placed on the skin to remove subcutaneous fat for aesthetic purposes.

However, there are many limitations to topical cryolipolysis. Treatments are longer and colder than needed to selectively target fat, as the cold temperature needs to diffuse through the skin to the underlying subcutaneous fat. Further, topical cryolipolysis relies on an applicator which greatly limits the anatomic areas that can be treated (i.e., an area can only be treated if it can be accommodated by a standard applicator). Topical cryolipolysis also lacks precision, as the cold diffuses in an uncontrolled manner over a broad area during lengthy treatment times that are necessary for topical application. Because cooling of the fat can only be achieved by diffusion of cold through the skin to the subcutaneous fat, this greatly limits the depth and amount of fat that can be removed.

### SUMMARY

The present invention provides a solution for making a slurry and a slurry. The slurry of the present invention can be used in injection cryolipolysis for fat removal, selective targeting of non-adipocyte, lipid rich tissue, and connective tissue remodeling, while avoiding non-specific hypertonic injury to tissue. The effects of cryolipolysis are enhanced by having a high percentage of ice in the slurry. Undesired effects, such as injury or inflammation at the injection site are reduced or avoided by adjusting or tuning components of the slurry or solution compositions, such as the osmolality, tonicity, pH, and temperature.

Injectable, biocompatible, sterile ice slurries present novel means for selectively cooling tissue in various therapeutic applications. Slurry injections enable cooling to be delivered at the injection site. Therapeutic slurry applications include but are not limited to fat removal for the selective targeting and removal of adipocytes or other lipid rich tissue for cosmetic purposes (for example, subcutaneous fat) and medical purposes (for example, visceral fat), stimulation of connective tissue remodeling, obstructive sleep apnea, and therapeutic hypothermia.

In the treatment of fat cells, once slurry is injected into a subject such as a human, the slurry causes cryolipolysis, or cell death, by freezing of fat cells. The percentage of ice in the slurry (referred to as ice coefficient) and temperature of the slurry are important, as these properties create the desired effect of fat removal. A temperature of the slurry should be cold enough to cause adipose cell death. However, the temperature should be warm enough to avoid tissue redness, blistering, tissue necrosis, and ulceration of surrounding tissue such as muscle and skin. For example, the temperature of the slurry may range from about −25° C. to about 10° C.

In some embodiments, the invention is a solution for generating a slurry comprising a solvent such as liquid water and one or more additives affecting the tonicity and/or flowability of the slurry and a slurry made from the solution.

In some embodiments, the invention is a slurry comprising liquid water, ice comprising from about 2% to about 70% by volume, and one or more additives affecting tonicity and/or flowability of the slurry.

A slurry of the present invention may be configured to be introduced to a subject such as a human via injection, therefore it may comprise additives that affect the ability of the slurry ice particles to flow through a delivery device such as a cannula. For example, ice particle shape, ice particle size and ice coefficient may be considered. Typically, additives that affect flowability include agents that affect the viscosity. Examples of biocompatible agents affecting viscosity include, for example, celluloses (i.e. carboxymethylcellulose, hydroxyethylcellulose, hydroxypropylmethylcellulose, methylcellulose), polyvinyl alcohol, polyvinylpyrrolidone, xanthan gum, polyethylene glycol, guar gum, locust bean gum, carrageenan, alginic acid, gelatin, acacia, and carbopol.

Additionally, because the slurry may be configured to be introduced to a human subject, additives that reduce tonicity alleviate adverse inflammatory and other effects at the injection site are included. Tonicity is a characteristic closely related to osmolality and osmolarity. Tonicity is the measure of an effective osmotic pressure gradient, or the measurement of osmotic pressure between two solutions. Osmolarity is the number of osmoles of solute per volume of solution (Osm/L), while osmolality is the number of osmoles of solute per mass of solvent (Osm/kg). Osmolarity and osmolality can be measured by any suitable method, such as by freezing point depression (FPD) and vapor point deficit (VPD). A solution is isotonic when the solution has the same osmotic pressure as some other solution, for example having the same osmotic pressure as a cell or body fluid. When the osmotic pressure is lower than a particular fluid, the solution is hypotonic. Similarly, when the osmotic pressure is higher than a particular fluid, the solution is hypertonic. Osmolality and osmolarity are important when considering compositions and formulations for injection into patients, such as humans. If osmolality/osmolarity are too high, the treated area may result in tissue redness, blistering, tissue necrosis, and ulceration. Furthermore, hypertonicity-induced effects of subcutaneous administration include enhanced site irritation and pain, enhanced tissue permeability, and possible tissue damage.

As such, the present invention tailors the osmolality to minimize these undesired effects associated with injection or administration of the slurry. In some embodiments, the slurry may have an osmolality of less than about 2,200 milli-Osmoles/kg. In some embodiments, the slurry has an osmolality of less than about 600 milli-Osmoles/kg. Examples of additives affecting tonicity include salts, cations, anions, polyatomic cations, polyatomic anions, sugars, and sugar alcohols. Increased levels of agents affecting tonicity (otherwise known as osmotically active compounds) enable the production of small, globular, injectable ice particles that are able to pass through a needle without clogging. However, the increased levels of agents affecting tonicity can result in hypertonic injury to tissue, as once they are injected into the body, the high osmolality of the slurry can dehydrate adjacent tissue. The present invention provides compositions in which the osmolality is well-tolerated by tissue.

Further, the pH of the slurry composition is important. The subject may experience pain at the injection site if the pH of the slurry composition is too high or too low. In an embodiment, the pH of slurry and solution compositions of the invention is about 4.5 to about 9.

Slurries and solutions of the invention can comprise biocompatible ingredients, such as water, ice, and additives recognized as safe for use in humans. For example, compositions of the invention further comprise one or more additives such as sodium chloride, glycerol, sodium carboxymethylcellulose (CMC), and others. The additives can be added to water prior to or during cooling and slurry production.

A slurry of the present invention may be administered by any suitable means. For example, the slurry may be injected through a delivery device such as a cannula. In some embodiments, the cannula is a needle. The particle size of the ice is important when choosing the gauge size of a needle. In some embodiments of the invention, each ice particle has a particle size of less than about 1 mm. In some instances, the particle size is less than about 0.25 mm. Slurry and solution compositions of the invention are suitable for use with a needle having a gauge size of about 8G to about 25G.

### BRIEF DESCRIPTION OF THE DRAWINGS

FIG. 1 shows a functional diagram of properties of solutions of the invention.

FIG. 2 shows a functional diagram of properties of slurries of the invention.

FIG. 3 shows an image of a slurry according to an embodiment of the invention.

FIG. 4 shows an image of a slurry according to an embodiment of the invention.

FIG. 5 shows an image of a slurry according to an embodiment of the invention.

### DETAILED DESCRIPTION

The present invention provides a solution for making a slurry and a slurry. In an embodiment, a solution or slurry of the present invention may be administered, e.g., injected, to a subject such as a human subject for removal of lipid rich tissue such as adipose tissue or fat. Typically, human subjects have fat deposits, namely deposits of subcutaneous fat under the skin and above muscle. Visceral fat deposits may be under the abdominal muscle and may surround organs in a human subject. Compositions of the invention rely on attributes such as flowability and tonicity in order to achieve optimal effectiveness and minimal pain and/or irritation of the treated area. For example, the invention enables the use of low-tonicity solutions and slurries, therefore allows for the minimization of pain, swelling, and other adverse effects that may be associated with high-tonicity solutions and slurries. Flowability is the ability of the slurry to flow through a device or within a subject. For example, flowability describes how easy it is for the slurry to move, either within the slurry generator, delivery device for administration such as a cannula, or within the body of a human patient. Flowability is dependent on several factors, including ice particle size, ice particle shape (as they relate to the configuration of the delivery device, for example, needle gauge) and viscosity.

In certain embodiments, the invention is a solution for making a slurry comprising liquid water and one or more additives. In certain embodiments, the invention is a slurry comprising liquid water, ice comprising from about 2% to about 70% by volume, and one or more additives. The one or more additives (and their respective concentrations) may be selected to affect the flowability and tonicity of the slurry administered to the subject.

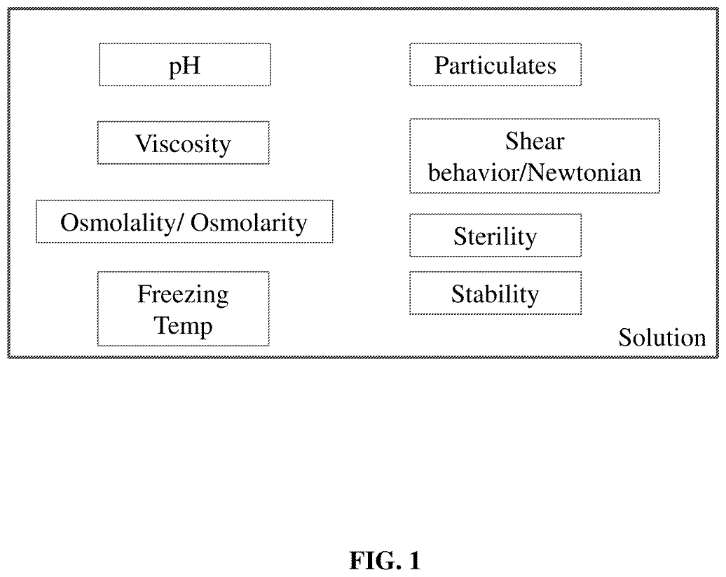

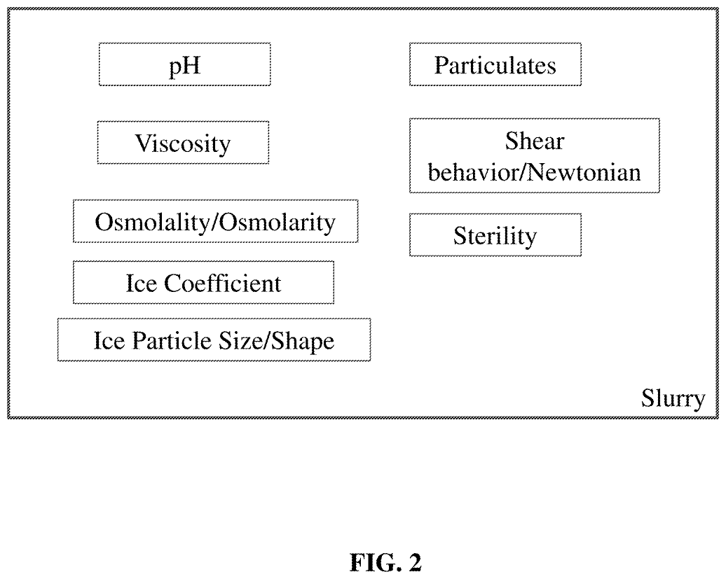

As shown in FIG. 1, various properties of the solution affecting flowability and tonicity include osmolarity/osmolality, viscosity, pH, particulates, shear behavior, and sterility. As shown in FIG. 2, various properties of the slurry affecting flowability and tonicity include those of the solution as well as ice coefficient and ice particle size and morphology.

Taking each property in turn, osmolarity is the number of osmoles of solute per volume of solution (Osm/L), while osmolality is the number of osmoles of solute per mass of solvent (Osm/kg). Osmolarity and osmolality can be measured by any suitable method, such as by freezing point depression (FPD) and vapor point deficit (VPD). Tonicity is a characteristic closely related to osmolality and osmolarity. Tonicity is the measure of an effective osmotic pressure gradient, or the measurement of osmotic pressure between two solutions. A solution is isotonic when the solution has the same osmotic pressure as some other solution, for example having the same osmotic pressure as a cell or body fluid. When the osmotic pressure is lower than a particular fluid, the solution is hypotonic. Similarly, when the osmotic pressure is higher than a particular fluid, the solution is hypertonic.

A consideration during solution formulation is the local and systemic tolerability of hypertonic slurries upon injection. Side effects depend on the degree of hypertonicity. Further, the sensation of pain is generally the worst in intramuscular injection, followed by subcutaneous injection and intravenous or intravascular injection. As an exemplary application, the present invention is directed to a solution and slurry suitable for injection in subcutaneous fat of a subject. Therefore, reducing or minimizing the sensation of pain, and the likelihood of adverse events, for the subject is factored into the present solution formulation.

Generally, solutions having an osmolality greater than approximately 300 mOsm/kg are hypertonic. Solutions having an osmolality lower than approximately 300 mOsm/kg are hypotonic. According to the invention, the osmolality can be adjusted based on the treatment and desired outcome. For example, the upper osmolality limit may be under about 1,500 mOsm/kg for intramuscular or subcutaneous injection. While for intravenous or intravascular injection, typically smaller volume injections, such as 100 mL or less, the upper limit may be under about 2,200 mOsm/kg. Further, the upper limit may be under about 600 mOsm/kg for larger volume injections, for example injections greater than 100 mL.

Studies have shown effects of hypertonicity on human patients. Based on the studies, the present invention is directed to solutions and slurries having an osmolality that will minimize the effects of inflammation in a human subject. For example, a study demonstrated that the subcutaneous injection should be less than 600 mOsm (Wang, 2015, Tolerability of hypertonic injectables, Int. J. Pharm., 490(1-2):308-315). Studies have reported a clinical trial having subcutaneous administration 845 mOsm/L, where patients received 1,000 mL over 12 hours daily for 7 days (Zaloga et al., 2017, Safety and efficacy of subcutaneous parenteral nutrition in older patients: a prospective randomized multicenter clinical trial, JPEN J Parenter Enteral Nutr, 41(7):1222-1227). Studies have also reported subcutaneous nutrition in the abdomen, chest, or thigh with 660 mOsm/L over a period of 5 days (Ferry et al., 1990, L′hypodermoclyse ou perfusion, Med et Hyg, 48:1533-1537) and 9.4 g nitrogen, 1,660 mOsm/L, pH 7, Kabi Pharmacia S A, Saint-Quentin-Yvelines, France subcutaneous infusion over 4 days in the abdomen (Ferry et al., 1997, Comparison of subcutaneous and intravenous administration of a solution of amino acids in older patients, J. Am. Geriatr. Soc., 45(7):857-860).

Hypertonicity-induced effects of subcutaneous administration include enhanced site irritation and pain, enhanced tissue permeability, and possible tissue damage. As such, the present invention tailors the osmolality to minimize inflammation effects (heat, redness, swelling, and pain) associated with injection or administration of the slurry. In some embodiments, a slurry of the present invention may comprise an osmolality of less than about 2,200 milli-Osmoles/kilogram. In some embodiments, the slurry may comprise an osmolality of less than about 600 milli-Osmoles/kilogram. By tailoring the osmolality of the slurry and solution compositions, the present invention reduces or minimizes pain associated with injection while remaining effective in providing a slurry at a temperature that results in targeted cell death of adipose tissue.

Additives that affect the viscosity of the slurry may affect the flowability of the slurry. Examples of biocompatible agents affecting viscosity include, for example, celluloses (i.e. sodium carboxymethylcellulose (CMC), hydroxyethylcellulose, hydroxypropylmethylcellulose, methylcellulose), polyvinyl alcohol, polyvinylpyrrolidone, xanthan gum, polyethylene glycol, guar gum, locust bean gum, carrageenan, alginic acid, gelatin, acacia, and carbopol. As an example, adding CMC or xanthan gum may increase the viscosity of the solution enough to improve flowability substantially relative to a sugarless solution.

To further mitigate pain associated with subcutaneous administration, extreme product pH and high buffer concentration may be avoided, an anesthetic agent may be used, the injection volume may be reduced, or a less painful route may be chosen. In some embodiments, the pH of the compositions is about 4.5 to about 9.

Particulates in the solution and/or slurry may be minimized for safety, but some particulates may be present to induce nucleation (nucleation is the initial process by which ice particles begin to form during slurry generation). As an example, more particulates may result in spontaneous nucleation, while fewer particulates may require induced nucleation to initiate generation of the slurry.

Shear behavior, or Newtonian behavior, is a property that affects various slurry generation processes/process parameters such as agitation and pump speed, and expression through a cannula, such as a needle. For example, ice particles may result in shear thickening, and CMC may result in shear thinning.

Sterility is a property related to safety, as the slurry is designed for injection into a human or non-human animal. In an embodiment, each ingredient of the solution is sterile. Accordingly, the solution or solution ingredients can be sterilized prior to generating the slurry by any suitable known sterilization techniques, such as autoclaving and UV sterilization techniques.

The freezing temperature is also a property of the solution. The freezing temperature is the temperature of the solution when there is some ice in the solution. To create a slurry, the solution must be cooled to generate ice particles. The freezing temperature can be affected by changing the additive concentrations, for example, salt and sugar levels. Moreover, the temperature of the slurry is an important property, as the slurry needs to be effective for treatment but safe for administering to the patient. In some embodiments, the slurry temperature can range from about −25° C. to about 10° C.

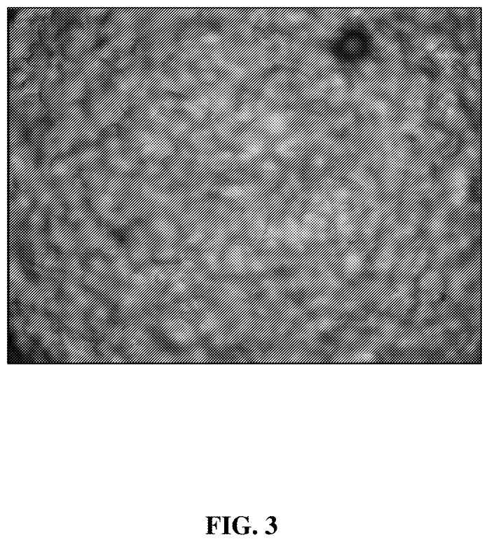

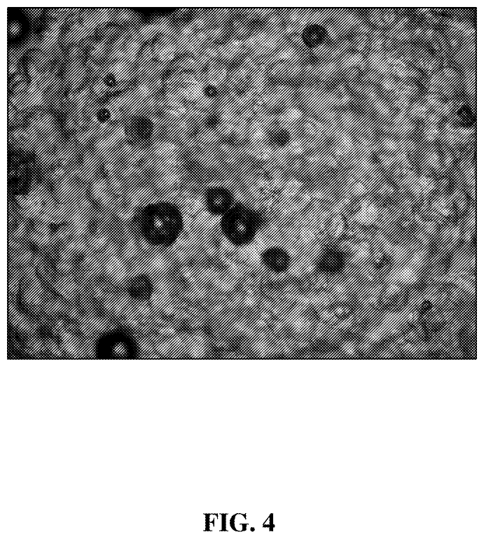

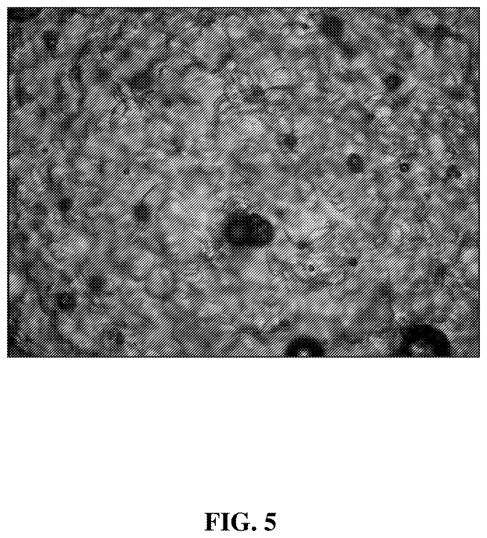

The ice coefficient is a property of the slurry that measures the amount of ice in the slurry, which affects at least the flowability of the slurry and effectiveness of treatment. In certain embodiments, the ice coefficient of the slurry is about 2% to about 70%. It is contemplated that more ice relates to more effectiveness per unit of injected volume, however the amount of ice can be balanced with maintaining the flowability of the slurry. FIG. 3 is an image of a slurry having an ice coefficient of 25%; FIG. 4 is an image of a slurry having an ice coefficient of 28%; and FIG. 5 is an image of a slurry having an ice coefficient of 22%.

Similarly, the ice particle size and morphology are properties of the slurry that affect at least flowability of the slurry and effectiveness of treatment. In preferred embodiments, the ice particles are sized to flow through a cannula of a desired size. For example, the cannula may be a needle, and the desired size may be determined based on gauge size of the needle. As an example, an ice particle size of about 100 μm may allow injection through a needle having an inside diameter of about 1.0 mm or smaller. Regarding the ice particle morphology or shape, the ice particles can be substantially rounded or globular.

In some embodiments, the one or more additives may be selected to impact one or more properties of the solution or slurry. In some embodiments, one or more additives may comprise a low molecular weight, therefore affecting certain properties while minimizing impact on other properties. For example, including more additives may improve the flowability, but also may increase the osmolarity and makes the solution more hypertonic.

In some embodiments, additives are inactive, biocompatible ingredients. Any suitable additive may be added to the solution or the slurry, including any substance/concentration in the FDA GRAS list, which is incorporated in its entirety herein.

Any acceptable concentration of one or more additives may be used in the present invention and may be selected based on the treatment. For example, for intradermal, subcutaneous, or intramuscular routes of administration, additives include sodium chloride (saline), glycerin/glycerol, dextrose, sodium CMC, xanthan gum, and polyethylene glycol. For example, acceptable concentrations of sodium chloride are about 0.9% for soft tissue use and about 2.25% for subcutaneous use, while acceptable concentrations of glycerin/glycerol are about 1.6% to about 2.0% for dermal use and about 15% for subcutaneous use. Further, acceptable concentrations of dextrose are about 5% w/v for intramuscular use and about 7.5% per unit dose for intramuscular-subcutaneous use. For example, acceptable concentrations of sodium CMC are about 0.75% for intralesional use, about 3% for intramuscular use, and about 0.5% to about 0.75% for soft tissue use. As another example, acceptable concentrations of xanthan gum are about 1% for intra-articular use in animal studies and about 0.6% for FDA ophthalmic use. Further, acceptable concentrations of polyethylene glycol, such as Polyethylene Glycol 3350, are about 2.0% to about 3.0% for FDA soft tissue use and about 4.42% for subcutaneous use.

In some embodiments, the salt is saline, a solution of sodium chloride (NaCl) in water. Saline is used in many medical applications and adds value as a source of water and electrolytes. Though saline has been shown to produce therapeutic benefits, care must be taken with the amount used in order to avoid injection pain. Studies have shown that pretreatment with 2% lidocaine attenuates pain response associated with hypertonic saline, and 4.8 mL over 600 sec intramuscular injection (1.0 cm to 2.0 cm depth) of hypertonic 5.8% saline has been shown to produce local and referred pain (Lei J, 2012, Variation of pain and vasomotor responses). Other examples of salts include potassium, calcium, magnesium, hydrogen phosphate, hydrogen carbonate.

In some embodiments, glycerol is an additive. For example, peritoneal dialysis has been used as an alternative to hemodialysis to help remove toxins from the body through infusing peritoneum with fluids called “dialysates”. Studies have shown that peritoneal dialysis with 0.6% amino acids and 1.4% glycerol are safe and well-tolerated in patients, and 48% glycerol has further been shown as an off label sclerosant (Van Biesen, 2004, A RCT with 0.6% amino acids/1.4% peritoneal dialysis solution and Dietzek, 2007, Sclerotherapy: Introduction to Solutions and Techniques).

In some embodiments, dextrose is an additive. Studies have demonstrated analgesic effects from 5% dextrose injections, therapeutic benefits for knee osteoarthritis with ACL laxity with 10% dextrose, and therapeutic benefits for myofascial pain syndrome with 5% dextrose (Maniquis-Smigel, 2017, Short Term Analgesic Effects of 5% Dextrose; Reeves & Hassanein 2000 and Reeves & Hassanein 2003; and Kim M Y, 1997).

In some embodiments, additives for affecting the viscosity include CMC and Xanthan Gum. Rabbit studies have examined intra-articular injections at 1% w/v (Guanying, 2017, Low molecular weight xanthan gum for the treatment of osteoarthritis). In another study, subjects received a 3.5 mL subcutaneous injection over 1 minute of placebo buffer (acetate) and sodium carboxymethylcellulose (Na CMC 7 mg/mL) at 250-350 mOsm. Pain was reported, but no necrosis was reported (Dias C, Tolerability High Volume Subcutaneous Injections, 2015). Further, CMC is used as key ingredient for polysaccharide dermal fillers, 20-45 mg/mL (Falcone S J, Novel Synthetic Dermal Fillers based on sodium carboxymethylcellulose, 2007).

In some embodiments, an additive may comprise a buffer to stabilize the pH. In some embodiments, an additive may comprise an emulsifier to create a smooth texture. In some embodiments, an additive may comprise a nanoparticle, for example, TiO2. The smaller sized particles in the solution may increase the number of nucleation sites, thus enabling creation of smaller ice particles. In some embodiments, an additive may comprise an agent configured as a coating for the ice particles which may prevent agglomeration during and after ice particle formation. In some embodiments, an additive may comprise IVF Synthetic Colloids at amounts of about 6.0% Hetastarch in about 0.9% sodium chloride; Poloxamer 188 at amounts of about 0.2% subcutaneous; Propylene Glycol at amounts of about 0.47% to about 1.4%; Benzyl Alcohol at amounts of about 0.9% to about 1.4%; gelatin at amounts of about 16%; and Icodextrin (used frequently in peritoneal dialysis) at amounts of about 7.5%.

The one or more additives affect the osmolarity of the solution and slurry. In certain embodiments, the slurry and solution compositions have an osmolarity lower than about 2,200 mOsm/L. In some embodiments, the osmolarity is less than about 600 mOsm/L. In such an embodiment, the slurry may comprise about 0.9% saline; about 1.0% to about 2.0% dextrose; about 1.0% to about 1.6% glycerol; less than about 0.5% sodium CMC; and less than about 0.6% xanthan gum. In one embodiment, the slurry composition may be about 500 mOsm/kg to about 700 mOsm/kg and comprise about 0.9% to about 1.4% saline; about 2.0% to about 4.0% dextrose; about 1.7% to about 2.0% glycerol; about 0.6% to about 1.0% sodium CMC; and about 0.6% to about 1.0% xanthan gum. In another embodiment, the slurry composition may be about 700 mOsm/kg to about 900 mOsm/kg and comprise about 1.5% to about 1.7% saline; about 5.0% to about 7.5% dextrose; about 3.0% to about 5.0% glycerol; about 1.0% to about 3.0% sodium CMC; and about 1.0% xanthan gum. In some embodiments, the slurry composition may be greater than about 1,000 mOsm/kg. In such an embodiment, the slurry may comprise about 1.8% to about 3.0% saline; about 10% dextrose; greater than about 5.0% glycerol; sodium CMC; and xanthan gum.

In some embodiments, the additives comprise one or more of a salt, a sugar, and a thickener. In an embodiment, the salt is NaCl at about 2.25% by mass or lower. In an embodiment, the sugar is glycerol at about 2% by mass or lower. In an embodiment, the thickener is CMC or Xanthan Gum at about 0.75% by mass or lower.

In some embodiments, the slurry can comprise an osmolality of less than about 2,200 mOsm/kg or lower, a temperature range of about −25° C. to about 10° C., an ice coefficient of about 2% to about 70%, and can pass through a needle having a gauge size of about 8G to about 25G for the selective targeting and removal of adipose tissue. In some embodiments, the slurry can comprise an osmolality of less than about 600 mOsm/kg, be in a temperature range of about −6° C. to about 0° C., and be able to pass through a needle with the minimum diameter of an about 8-25G gauge size for the selective targeting and removal of adipose tissue.

When considering solutions and slurries of the invention, properties such as osmolality/osmolarity, viscosity, pH, particulates, shear behavior, sterility, ice coefficient, ice size and ice morphology can be affected based on the additives (and their respective amounts) chosen, and one or more properties may be affected while one or more other properties remains unaffected.

A slurry can be generated from a solution using any of the systems and methods disclosed in U.S. Provisional Patent Application Ser. No. 62/743,830 and U.S. Provisional Patent Application Ser. No. 62/743,908, which are both incorporated by reference in their entirety herein. A slurry can be used to a treat a subject using any of the methods disclosed in U.S. Provisional Patent Application Ser. No. 62/741,286 which is incorporated by reference in its entirety herein.

### EXAMPLES

### Example 1: Slurry

In this example, the slurry comprises a sterile water-based solution containing the following additives included for the purpose of freezing point depression, ensuring globular ice particle shape, appropriate flow dynamics and viscosity: sodium chloride, glycerol, and sodium carboxymethylcellulose (CMC). The additives were selected due to their safety and tolerability profiles, and all concentrations are substantially lower than commercially available approved products contained in the FDA's GRAS List, which for subcutaneous injections dosing limits are: 2.25% sodium chloride, 2% glycerol, and 0.75% sodium carboxymethylcellulose. Table 1 summarizes the exemplary slurry additives.

TABLE 1
Solution/Slurry Additives

| | | FDA Inactive | |
|---|---|---|---|
| | | Ingredient | |
| Inactive | GRAS? | (Max Dose: | |
| Ingredient | Type I | Subcutaneous) | Additive Function |
| Sodium | Y (1979) | 2.25% | Freezing Point Depression |
| Chloride | | | Ideal Ice Particle Geometry |
| | | | Flow Dynamics |
| Glycerol | Y (1975) | 2% | Freezing Point Depression |
| | | | Ideal Ice Particle Geometry |
| | | | Flow Dynamics |
| | | | Viscosity |
| Sodium | Y (1973) | 0.75% | Flow Dynamics |
| CMC | | | Viscosity |

### Example 2: Rabbit Model

A rabbit study was undertaken to identify the safety and tolerability limits of increasingly concentrated or dilute variants of the sterile excipient solution. The concentration of CMC was held constant, as increased levels of CMC impact solubility. CMC does not contribute to freezing point depression or ice particle geometry, which are two key parameters in making an injectable slurry.

Each adult New Zealand White rabbit (Oryctolagus cuniculus) weighing 3-4 kg used in the study was injected with 3.6 mL of a candidate slurry into the intrascapular fat pad. Human treatment may comprise injecting 30 mL of the slurry per site for multiple sites. For example, human treatment may comprise four sites for a total of 120 mL per treatment. To correspond to the 120 mL treatment for a human reference weight of 60 kg, a 3.6 mL injection was chosen for a 1.8 kg rabbit. Primary endpoints are incidence of adverse effects as observed by gross photography and histologic imaging. Blood samples and Body Condition Scores (BCS) were also obtained.

Solutions having osmolalities listed in Table 2 were injected into the intrascapular space into the fat pad. As shown in Table 2, eight different solutions, each with a different osmolality, were injected into the rabbits. Each solution represented a dilution or concentration of the test solution, and each solution was increasingly hypertonic.

TABLE 2
Osmolality of Solutions used in Testing Groups

| Testing Group | 1 | 2 | 3 | 4 | 5 | 6 | 7 | 8 |
|---|---|---|---|---|---|---|---|---|
| Osmolality (mOsm/kg) | 510 | 686 | 865 | 1,048 | 1,422 | 2,214 | 3,068 | 3,993 |

A rabbit model was chosen given its exquisite sensitivity and routine use in assessing irritant potential. Rabbits are the recommended model for testing acute dermal irritation. Previous research in swine and rodent models showed safety and tolerability of solutions in excess of 1,400 mOsm/L.

Each rabbit received a 3.6 mL subcutaneous injection of test solution into the intrascapular fat pad to simulate the bolus slurry injection that may be used in each of three sites in human testing. The 3.6 mL injection in 3-4 kg rabbits is equivalent to a 54-72 mL injection in a 60 kg adult, which is sufficient to simulate the total injection volume for any systemic effects change in body weight, blood tests, and BCS.

Injections were done in a dose escalation design. All animals tolerated the procedure well, and quickly (within 5-10 minutes) returned to normal behavior when returned to their cage. At time of completion (90 minutes post-initial injection), no immediate adverse effects were noted. Transient mild bruising and erythema was seen in Groups 6 and 7 at 24 hours post-injection and were resolved by 48 hours post-injection. Mild erythema was noted in Group 8, and the erythema persisted until the time of sacrifice, one week after injection. This work demonstrated that solutions comprising an osmolality of about 2200 mOsm/kg or less are well tolerated and may be suitable for injectable slurries.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

1. A slurry comprising liquid water, about 20% to about 70% ice particles by volume, sodium chloride, glycerol in an amount of 2 mass % or less, and sodium carboxymethylcellulose,
wherein the slurry has a pH from about 4.5 to about 9.

2. The slurry of claim 1, wherein the ice particles substantially rounded or globular, and capable of flowing through a cannula.

3-5. (canceled)

6. The slurry of claim 1, wherein the slurry has an osmolality of less than 2,200 milli-Osmoles/kilogram.

7. The slurry of claim 1, wherein the slurry has an osmolality of less than 600 milli-Osmoles/kilogram.

8. The slurry of claim 1, wherein the slurry comprises a temperature from about −25° C. to about 10° C.

9. The slurry of claim 1, wherein the slurry has a temperature from about −6° C. to about 0° C.

10. (canceled)

11. The slurry of claim 1, wherein the ice particles have a particle size of less than 1 mm.

12. The slurry of claim 11, wherein the ice particles have a particle size of less than 0.25 mm.

13. (canceled)

14. The slurry of claim 1, further comprising one or more additives selected from the group consisting of dextrose, xanthan gum, glycerin, polyethylene glycol, cellulose, polyvinyl alcohol, polyvinylpyrrolidone, guar gum, locust bean gum, carrageenan, alginic acid, gelatin, acacia, acrylic acid crosslinked with allyl sucrose, and acrylic acid crosslinked with allyl pentaerthyritol.

15-34. (canceled)
